(19) United States
(12) Patent Application Publication
(10) Pub. No.: US 2023/0364367 A1
(43) Pub. Date: Nov. 16, 2023

(54) SYSTEM AND METHOD FOR IMPROVED FLOW DETECTION DURING HIGH FREQUENCY VENTILATION

(71) Applicants: Erich J. Barischoff, South Salem, OR (US); Kori Dunn, Grand Island, NY (US); Paul K. Oetinger, Gasport, NY (US)

(72) Inventors: Erich J. Barischoff, South Salem, OR (US); Kori Dunn, Grand Island, NY (US); Paul K. Oetinger, Gasport, NY (US)

(21) Appl. No.: 17/741,578

(22) Filed: May 11, 2022

(51) Int. Cl. A61M 16/00; G01F 15/00; A61M 16/20

(52) U.S. Cl. CPC A61M 16/0096; G01F 15/005; A61M 16/0003; A61M 16/208; A61M 16/024; A61M 16/0051; A61M 2202/0275; A61M 2205/581; A61M 2016/0036

(57) ABSTRACT

A gas delivery system and method are provided that include a therapeutic gas delivery device, and a high-frequency ventilator delivering a breathing gas to a respiratory circuit. The therapeutic gas delivery system also includes a flow sensor assembly having a single interior chamber. The flow sensor assembly includes a flow sensor connected with the therapeutic gas delivery device and configured to measure a flow rate of the breathing gas. The flow sensor has a sensor gas inlet and a sensor gas outlet. The flow sensor assembly further includes a one-way flow valve disposed downstream of the sensor gas outlet. The one-way flow valve includes a valve gas inlet and a valve gas outlet. Inner diameters of the valve gas inlet and the valve gas outlet are greater than or equal to an inner diameter of at least one of the sensor gas inlet and the sensor gas outlet.

### FIELD

The present disclosure relates generally to high frequency jet ventilation (HFJV) systems, and more particularly, to improved flow detection in an HFJV system.

### BACKGROUND

HFJV delivers rapid pulses of breathing gas to the lungs of a patient. When HFJV is utilized in conjunction with inhaled therapeutic gas (e.g., nitric oxide (NO) therapy), a flow sensor is typically inserted into a respiratory circuit of the high-frequency jet ventilator. This flow sensor is utilized to determine the flow rate of the breathing gas so that the therapeutic gas (e.g., NO) may be dosed accordingly. However, due to the nature of the pulses delivered by the high-frequency jet ventilator, the measured flow rate may be inaccurate or may not be detected at all. Consequently, therapeutic gas dosing may also be inaccurate, and alarms may be triggered on the therapeutic gas delivery device when a flow rate is not detected or when the delivered dose of therapeutic gas exceeds a set limit.

A small barbed check valve, or one-way flow valve, which fits existing tubing (e.g., 3/16″ (4.8 mm) tubing), may be disposed directly upstream of a humidifier of the high-frequency ventilator and downstream of a flow sensor when HFJV is used in conjunction with inhaled therapeutic gas. However, this small check valve does not minimize undesirable dose fluctuations or flow rate alarms that may appear on the therapeutic gas delivery device under certain HFJV operating conditions. Moving this small barbed check valve closer to the flow sensor also does not cure these defects.

### SUMMARY

According to an embodiment, a gas delivery system is provided that includes a therapeutic gas delivery device, and a high-frequency ventilator delivering a breathing gas to an inspiratory limb of a respiratory circuit. The therapeutic gas delivery system also includes a flow sensor assembly in the inspiratory limb and having a single interior chamber. The flow sensor assembly includes a flow sensor connected with the therapeutic gas delivery device and configured to measure a flow rate of the breathing gas from the high-frequency ventilator. The flow sensor has a sensor gas inlet and a sensor gas outlet. The flow sensor assembly further includes a one-way flow valve disposed downstream of the sensor gas outlet in the flow sensor assembly. The one-way flow valve includes a valve gas inlet and a valve gas outlet. Inner diameters of the valve gas inlet and the valve gas outlet are greater than or equal to an inner diameter of at least one of the sensor gas inlet and the sensor gas outlet.

According to an embodiment, a flow sensor assembly is provided that includes a body section having an interior chamber, a gas inlet to the body section, and a gas outlet to the body section. The flow sensor assembly also includes a flow sensor in the body section, configured to measure a gas flow rate and having a sensor gas inlet and a sensor gas outlet. The flow sensor assembly further includes a one-way flow valve in the body section and disposed between the sensor gas outlet and the gas outlet. The one-way flow valve includes a valve gas inlet and a valve gas outlet. Inner diameters of the valve gas inlet and the valve gas outlet are greater than or equal to an inner diameter of at least one of the sensor gas inlet and the sensor gas outlet.

According to an embodiment, a method is provided for delivering therapeutic gas. A high-frequency ventilator delivers a breathing gas to an inspiratory limb of a respiratory circuit. A flow rate of the breathing gas from the high-frequency ventilator is measured by a flow sensor assembly in the inspiratory limb. The flow sensor assembly includes a flow sensor connected with a therapeutic gas delivery device and has a sensor gas inlet and a sensor gas outlet. The flow sensor assembly also includes a one-way flow valve disposed downstream of the sensor gas outlet and includes a valve gas inlet and a valve gas outlet. Inner diameters of the valve gas inlet and the valve gas outlet are greater than or equal to an inner diameter of at least one of the sensor gas inlet and the sensor gas outlet. Based on the measured flow rate, the therapeutic gas delivery device doses therapeutic gas to be combined with the breathing gas in the inspiratory limb of the respiratory circuit.

### BRIEF DESCRIPTION OF THE DRAWINGS

The above and other aspects, features, and advantages of certain embodiments of the present disclosure will be more apparent from the following detailed description, when taken in conjunction with the accompanying drawings, in which:

FIG. 1 is a diagram illustrating an HFJV system utilized in conjunction with inhaled therapeutic gas, according to an embodiment;

FIG. 2 is a diagram illustrating the flow sensor assembly of FIG. 1, according to an embodiment;

FIG. 3 is a flowchart illustrating a method for delivering a therapeutic gas in conjunction with an HFJV system, according to an embodiment; and

FIG. 4 is a block diagram illustrating a controller for controlling a therapeutic gas delivery device, according to an embodiment.

### DETAILED DESCRIPTION

Hereinafter, embodiments of the present disclosure are described in detail with reference to the accompanying drawings. It should be noted that the same elements will be designated by the same reference numerals although they are shown in different drawings. In the following description, specific details such as detailed configurations and components are merely provided to assist with the overall understanding of the embodiments of the present disclosure. Therefore, it should be apparent to those skilled in the art that various changes and modifications of the embodiments described herein may be made without departing from the scope of the present disclosure. In addition, descriptions of well-known functions and constructions are omitted for clarity and conciseness. The terms described below are terms defined in consideration of the functions in the present disclosure, and may be different according to users, intentions of the users, or customs. Therefore, the definitions of the terms should be determined based on the contents throughout this specification.

The present disclosure may have various modifications and various embodiments, among which embodiments are described below in detail with reference to the accompanying drawings. However, it should be understood that the present disclosure is not limited to the embodiments, but includes all modifications, equivalents, and alternatives within the scope of the present disclosure.

Although the terms including an ordinal number such as first, second, etc. may be used for describing various elements, the structural elements are not restricted by the terms. The terms are only used to distinguish one element from another element. For example, without departing from the scope of the present disclosure, a first structural element may be referred to as a second structural element. Similarly, the second structural element may also be referred to as the first structural element. As used herein, the term “and/or” includes any and all combinations of one or more associated items.

The terms used herein are merely used to describe various embodiments of the present disclosure but are not intended to limit the present disclosure. Singular forms are intended to include plural forms unless the context clearly indicates otherwise. In the present disclosure, it should be understood that the terms “include” or “have” indicate the existence of a feature, a number, a step, an operation, a structural element, parts, or a combination thereof, and do not exclude the existence or probability of the addition of one or more other features, numerals, steps, operations, structural elements, parts, or combinations thereof.

Unless defined differently, all terms used herein have the same meanings as those understood by a person skilled in the art to which the present disclosure belongs. Terms such as those defined in a generally used dictionary are to be interpreted to have the same meanings as the contextual meanings in the relevant field of art and are not to be interpreted to have ideal or excessively formal meanings unless clearly defined in the present disclosure.

In order to improve the accuracy of a flow rate measurement by a flow sensor in a respiratory circuit during HFJV, a one-way flow valve may be disposed downstream of the flow sensor. The one-way flow valve prevents backflow into the flow sensor, leading to improved accuracy of flow rate measurement and therapeutic gas delivery, as well as a reduction in the frequency of flow related alarms at the therapeutic gas delivery device. The inner diameters of an inlet and an outlet of the one-way flow valve may be similar to or larger than the inner diameter of the flow sensor inlet and/or outlet in order to improve flow detection. For example, a 22 mm one-way flow valve disposed downstream of a flow sensor achieves such results.

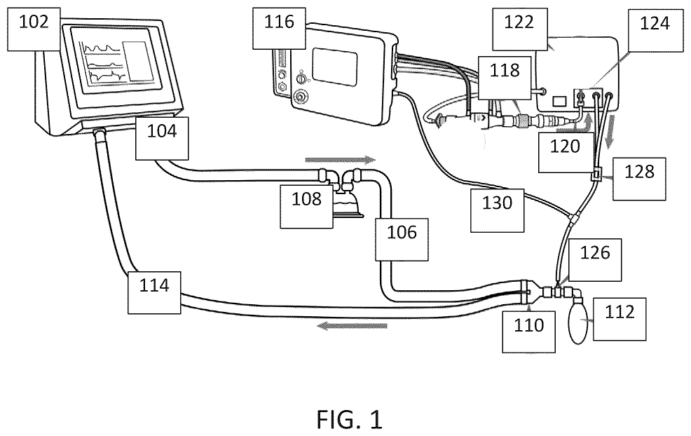

FIG. 1 is a diagram illustrating an HFJV system utilized in conjunction with inhaled therapeutic gas, according to an embodiment. The therapeutic gas may be NO. Specifically, a therapeutic gas delivery system (e.g., NOxBOXᵢ® available from NOxBOX Ltd.) is utilized with a high-frequency jet ventilator (e.g., Bunnell LifePulse®), and a one-way flow valve and required fittings are shown downstream of a flow sensor. As seen therein, a ventilator 102 includes an inspiratory port 104 through which breathing gas is provided to a ventilator inspiratory limb 106. The ventilator inspiratory limb 106 includes a ventilator humidifier 108 and ends at an inlet end of a Y-piece 110 before a patient end 112. The Y-piece 110 also includes an outlet end that is the beginning of a ventilator expiratory limb 114, which ends at ventilator 102.

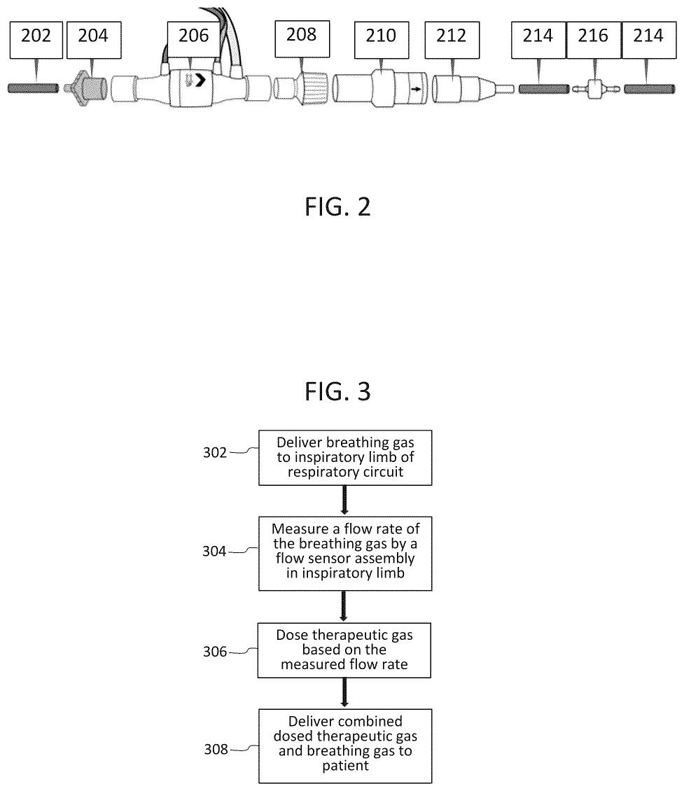

A therapeutic gas delivery system and mixer head unit 116 (e.g., NOxBOXᵢ® and NOxMixer™ system head unit available from NOxBOX Ltd.) is connected to a flow sensor assembly 118. The flow sensor assembly 118 and its connections are described in greater detail below with respect to FIG. 2. An outlet end of the flow sensor assembly 118 is connected, via gas-out tubing 120, to an inlet of a high-frequency jet ventilator 122 (e.g., Bunnell LifePulse®). Specifically, the gas-out tubing is connected to an inlet of a humidifier 124 of the high-frequency jet ventilator 122. The high-frequency jet ventilator 122 is connected to an inlet end of the flow sensor assembly 118. Gas-out tubing connects an outlet of the humidifier 124 of the high-frequency jet ventilator 122 to an adaptor 126 at a base end of the Y-piece 110. Before reaching the adaptor 126, gas-out tubing is provided through a patient box 128. Accordingly, the above-described flow is referred to as a high-frequency ventilator inspiratory limb of the respiratory circuit to which the high-frequency jet ventilator 122 provides breathing gas. A sample line 130 (e.g., NOxBOXᵢ® system sample line) connects the tubing from the adaptor 126 to the therapeutic gas delivery system and mixer head unit 116.

FIG. 2 is a diagram illustrating the flow sensor assembly of FIG. 1, according to an embodiment. Gas-out tubing 202 connects the high frequency jet ventilator 122 to an adaptor 204 (e.g., 15M—4.5 mm adaptor) at an inlet end of the flow sensor assembly 118. The flow sensor assembly 118 includes a single body section with an interior chamber. An outlet end of the adaptor 204 is connected to an inlet end of a flow sensor 206 (e.g., NOxFLOW sensor).

The flow sensor assembly 118, and more specifically, the flow sensor 206, also has connections to the therapeutic gas delivery system and mixer head unit 116. The flow sensor 206 may be fluidly, electrically, and/or wirelessly connected to the therapeutic gas delivery system and mixer head unit 116. For example, two differential pressure lines and a NO dosing line may connect the flow sensor 206 to the therapeutic gas delivery system and mixer head unit 116. The flow sensor 206 may be embodied as a pneumotach, a hot wire anemometer, or an ultrasonic flow meter.

The flow sensor 206 provides one or more sensed parameters to the therapeutic gas delivery system and mixer head unit 116. These sensed parameters include at least a flow rate measured at the flow sensor 206. The therapeutic gas delivery system and mixer head unit 116 may include a processor that stores the measured flow rate from the flow sensor 206. The therapeutic gas delivery system and mixer head unit 116 may provide an alarm that alerts a user via audible and/or visual means when backflow, high frequency flow, or another anomaly is ascertained by the processor using the measured flow rate detected by the flow sensor 206. An outlet end of the flow sensor 206 is connected to an inlet end of a connector 208.

An outlet end of the connector 208 is connected to an inlet end of a first one-way flow valve 210. The first one-way flow valve 210 may be embodied as a diaphragm one-way flow valve, a duckbill one-way flow valve, a disc one-way flow valve, or a ball one-way flow valve. The first one-way flow valve 210 prevents backflow into the flow sensor 206, improving the accuracy of flow detection. Inlet and outlet ends of the first one-way flow valve 210 have inner diameters that are greater than or similar to the inlet end and/or outlet end of the flow sensor 206. The first one-way flow valve 210 may be embodied as a 22 mm one-way flow valve.

The outlet end of the first one-way flow valve 210 is connected to a straight connector 212 (e.g., a straight connector 22M—6 mm oxygen stem), having an outlet end connected to the gas-out tubing 120. A second one-way flow valve 216 is disposed along a length of the gas-out tubing 120.

Embodiments of flow sensor assembly reduce backflow into a flow sensor, improve flow rate measurements, reduce alarms, and lessen fluctuations in doses of therapeutic gas by the therapeutic gas delivery system. Such advantages are provided at a small economic cost of adding the one-way flow valve in the flow sensor assembly. According to another embodiment, the flow sensor 206 may include a built-in one-way flow valve, such that no extra fittings (e.g., connectors) are required.

The flow sensor assembly may be used with any type of ventilation and is not limited to HEW. For example, the flow sensor assembly may be utilized with high-frequency oscillatory ventilation, high-frequency percussive ventilation, and high-frequency positive pressure ventilation. The entire flow sensor assembly may be disposable or reusable. The flow sensor assembly may be used with any application that requires inline flow detection and is not limited to NO therapy.

As described above, the therapeutic gas delivery device may be able to detect backflow or pulsating flow based on rapid changes in the flow rate detected by the flow sensor. The device may display an alert or message on a display that informs the user to insert a one-way flow valve downstream of the flow sensor, or to check that an installed one-way flow valve is functioning properly (e.g., check that the one-way flow valve does not have a folded or distorted diaphragm).

Referring now to FIG. 3, a flowchart illustrates a method for delivering a therapeutic gas in conjunction with an HFJV system, according to an embodiment. At 302, a breathing gas is delivered to an inspiratory limb of a respiratory circuit by a high frequency ventilator. At 304, a flow rate of the breathing gas from the high-frequency ventilator is measured by a flow sensor assembly in the inspiratory limb. The flow sensor assembly includes a flow sensor connected with a therapeutic gas delivery device and has a sensor gas inlet and a sensor gas outlet. The flow sensor assembly also includes a one-way flow valve disposed downstream of the sensor gas outlet and includes a valve gas inlet and a valve gas outlet. Inner diameters of the valve gas inlet and the valve gas outlet are greater than or equal to an inner diameter of at least one of the sensor gas inlet and the sensor gas outlet. When properly operational, the one-way flow valve prevents gaseous backflow to the flow sensor.

At 306, based on the measured flow rate, the therapeutic gas delivery device doses therapeutic gas to be combined with the breathing gas in the inspiratory limb of the respiratory circuit. The flow sensor provides one or more sensed parameters, including at least the measured flow rate, to the therapeutic gas delivery device. An alarm may be issued by the therapeutic gas delivery device when backflow, high frequency flow, or another anomaly is ascertained by a processor of the therapeutic gas delivery device using the measured flow rate detected by the flow sensor. At 308, the combined dosed therapeutic gas and the breathing gas are delivered to a patient.

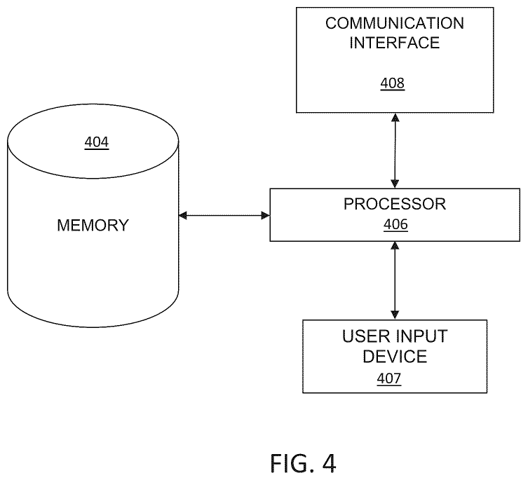

FIG. 4 is a block diagram illustrating a controller for controlling a therapeutic gas delivery device, according to an embodiment. The controller may be embodied as a programmable logic controller (PLC). The controller may include at least one user input device 407 and a memory 404 for storing sensed parameters, including at least measured flow rates. The apparatus also includes a processor 406 for determining doses based on the measured flow rates, and when to issue an alarm and/or provide instructions based on the measured flow rates. Additionally, the apparatus may include a communication interface 408 for outputting alarms and/or instructions to a user, and for receiving input from the user in response to instructions.

Although certain embodiments of the present disclosure have been described in the detailed description of the present disclosure, the present disclosure may be modified in various forms without departing from the scope of the present disclosure. Thus, the scope of the present disclosure shall not be determined merely based on the described embodiments, but rather determined based on the accompanying claims and equivalents thereto.

1. A gas delivery system comprising:
a therapeutic gas delivery device;
a high-frequency ventilator delivering a breathing gas to an inspiratory limb of a respiratory circuit; and
a flow sensor assembly disposed in the inspiratory limb and comprising:
a flow sensor connected with the therapeutic gas delivery device and configured to measure a flow rate of the breathing gas from the high-frequency ventilator, wherein the flow sensor has a sensor body section having an interior chamber, a sensor gas inlet and a sensor gas outlet; and
a one-way flow valve disposed downstream of the sensor gas outlet in the flow sensor assembly, wherein the one-way flow valve comprises a valve gas inlet and a valve gas outlet, and wherein diameters of the valve gas inlet and the valve gas outlet are greater than or equal to a diameter of at least one of the sensor body section, the sensor gas inlet and the sensor gas outlet.

2. The gas delivery system of claim 1, wherein the high frequency ventilator comprises a high frequency jet ventilator, a high frequency oscillatory ventilator, a high-frequency percussive ventilator, or a high-frequency positive pressure ventilator.

3. The gas delivery system of claim 1, wherein the flow sensor comprises a pneumotach, a hot wire anemometer, or an ultrasonic flow meter.

4. The gas delivery system of claim 1, wherein the flow sensor is connected with the therapeutic gas delivery device via at least one of a fluid connection, an electrical connection, and a wireless connection.

5. The gas delivery system of claim 1, wherein the therapeutic gas delivery device comprises a processor, and the flow sensor assembly provides one or more sensed parameters to the processor.

6. The gas delivery system of claim 5, wherein the one or more sensed parameters comprise at least the measured flow rate from the flow sensor.

7. The gas delivery system of claim 6, wherein the processor is configured to provide at least one of an audio alarm and a visual alarm when backflow, high frequency flow, or another anomaly is ascertained by the processor using the measured flow rate detected by the flow sensor.

8. The gas delivery system of claim 5, wherein the processor is configured to provide instruction to a user to confirm a presence or condition of the one-way flow valve when the backflow, the high frequency flow, or the other anomaly is ascertained by the processor.

9. The gas delivery system of claim 1, wherein the therapeutic gas is nitric oxide.

10. The gas delivery system of claim 1, wherein the one-way flow valve is a diaphragm one-way flow valve, a duckbill one-way flow valve, a disc one-way flow valve, or a ball one-way flow valve.

11. The gas delivery system of claim 1, wherein the flow sensor is directly connected to the one-way flow valve via a first connector, and the one-way flow valve is directly connected to gas-out tubing via a second connector.

12. A flow sensor assembly comprising:
a gas inlet;
a gas outlet;
a flow sensor disposed between the gas inlet and the gas outlet and configured to measure a gas flow rate, the flow sensor having a sensor body section having an interior chamber, a sensor gas inlet and a sensor gas outlet; and
a one-way flow valve disposed between the sensor gas outlet and the gas outlet, wherein the one-way flow valve comprises a valve gas inlet and a valve gas outlet, and wherein a diameter of the valve gas inlet and a diameter of the valve gas outlet are greater than or equal to a diameter of at least one of the sensor body section, the sensor gas inlet, and the sensor gas outlet.

13. The flow sensor assembly of claim 12, wherein the flow sensor comprises a pneumotach, a hot wire anemometer, or an ultrasonic flow meter.

14. The flow sensor assembly of claim 12, wherein the flow sensor is connected with a therapeutic gas delivery device via at least one of a fluid connection, an electrical connection, and a wireless connection.

15. The flow sensor assembly of claim 14, wherein the flow sensor provides the measured flow rate to the therapeutic gas delivery device.

16. The flow sensor assembly of claim 12, wherein the one-way flow valve is a diaphragm one-way flow valve, a duckbill one-way flow valve, a disc one-way flow valve, or a ball one-way flow valve.

17. The flow sensor assembly of claim 12, wherein the flow sensor is directly connected to the one-way flow valve via a first connector, and the one-way flow valve is directly connected to gas-out tubing via a second connector.

18. A method for a delivering therapeutic gas, the method comprising steps of:
providing a therapeutic gas delivery device;
delivering, by a high-frequency ventilator, a breathing gas to an inspiratory limb of a respiratory circuit;
measuring a flow rate of the breathing gas from the high-frequency ventilator
measuring a flow rate of the breathing gas from the high-frequency ventilator using a flow sensor assembly; and
dosing a therapeutic gas from the therapeutic gas delivery device to the inspiratory limb of the respiratory circuit based on the measured flow rate of the breathing gas;
wherein the flow sensor assembly comprises:
a flow sensor connected with a therapeutic gas delivery device and having a sensor body section having an interior chamber, a sensor gas inlet and a sensor gas outlet; and
a one-way flow valve disposed downstream of the sensor gas outlet and comprising a valve gas inlet and a valve gas outlet, wherein a diameter of the valve gas inlet and a diameter of the valve gas outlet are greater than or equal to a diameter of at least one of the sensor body section, the sensor gas inlet and the sensor gas outlet.

19. The method of claim 18, further comprising steps of:
providing the measured flow rate from the flow sensor assembly to the therapeutic gas delivery device; and
issuing an alarm, by the therapeutic gas delivery device, when backflow, high frequency flow, or another anomaly is ascertained by the therapeutic gas delivery device using the measured flow rate detected by the flow sensor assembly.

20. The method of claim 18, further comprising the steps of:
combining the dosed therapeutic gas from the therapeutic gas delivery device with the breathing gas in the inspiratory limb of the respiratory circuit; and
delivering the combined dosed therapeutic gas and the breathing gas to a patient.
